(19) United States
(12) Patent Application Publication
(10) Pub. No.: US 2010/0256175 A1
(43) Pub. Date: Oct. 7, 2010

(54) TREATMENT FOR DRUG ADDICTION

(76) Inventor: D. Bommi Bommannan, 1977 Wimbledon Place, CA (US)

Correspondence Address: D. BOMMI BOMMANNAN, 2251 Grant Road, Suite B, LOS ALTOS, CA, 94024, US

(21) Appl. No.: 12/754,559

(22) Filed: Apr. 5, 2010

Related U.S. Application Data
(60) Provisional application No. 61/166,516, filed on Apr. 3, 2009.

(51) Int. Cl. A61K 31/4355; A61K 31/135; A61P 25/30; G01N 33/68

(52) U.S. Cl. 514/282; 514/648; 436/86

(57) ABSTRACT

The invention discloses a method of treatment for drug addiction, comprising administering a therapeutic agent to a patient. The therapeutic agent is delivered in an inactive state and is converted into its active form in the presence of a narcotic. The conversion produces at least one unpleasant side effect for the patient. The therapeutic agent is implanted in the form of a depot, and maybe a prodrug or microspheres. The agent may be injectable or may be packaged in the form of bioerodible sticks. The agent may also be delivered via a surgical method. The disclosed method can also be used to detect the consumption of a banned substance.

### CROSS-REFERENCE TO RELATED APPLICATIONS

This application claims priority to U.S. Provisional Application Ser. No. 61/166,516, filed on Apr. 3, 2009, titled “Drug Addiction Treatment”.

### FIELD OF THE INVENTION

This invention relates to treatment of drug addiction, and in particular to methods for treating drug addiction without reliance on patient compliance.

### DESCRIPTION OF THE RELATED ART

Drug abuse and drug addiction are a major problem confronting societies all over the world. According to the World Drug Report 2008, about 5% of the world population uses illicit drugs and in 0.6% of the world population, drug use is a problem. In the USA, according to the Substance Abuse and Mental Health Services Administration's (SAMHSA's) National Survey on Drug Use and Health in 2006, 23.6 million persons aged 12 or older needed treatment for an illicit drug or alcohol abuse problem (9.6 percent of the persons aged 12 or older). Of these, only 2.5 million—10.8 percent of those who needed treatment—received it at a specialty facility.

Drug addiction causes enormous loss of productive manpower all over the world and imposes great costs on governments and societies in terms of treatment support, insurance payouts, and spending on prevention and de-addiction programs. Furthermore, abuse of illicit drugs such as narcotics poses a great threat to society in the form of increased interpersonal conflicts, loss of productivity and livelihood, loss of family coherence, psychological impact and loss of support for children, etc.

Federally administered drug addiction treatment programs are a national priority for the US Government. The National Institutes of Health estimates that when savings related to health care are included, total savings can exceed costs by a ratio of 12 to 1. Further, money invested in addiction treatment yields significant returns when considering factors such as reduced drug-related crime, criminal justice costs, and theft. Other reported benefits to the individual and society have been through reductions in interpersonal conflicts, workplace inefficiencies and drug-related accidents.

Drug addiction treatment involves administering to the addict a periodic dose of a treatment medication, such as methadone, levomethadyl acetate (also known as levo-a-acetylmethadol (LAAM)), and naltrexone for individuals addicted to opiates. These medications are used both for detoxification and for preventing or antidoting the effects of narcotics. The active agent in the treatment medication, such as naltrexone, acts by blocking the receptor in the patient's system corresponding to the addictive drug, which makes ingestion of the addictive drug ineffective.

Opiate addiction is treated through injections and commonly through oral tablets, but more recently through implants containing naltrexone, for example. A non-biodegradable implant containing a formulation with naltrexone is disclosed in U.S. Pat. No. 6,203,813, while an implant with hydromorphone is disclosed in U.S. Pat. No. 5,633,000. Sustained release formulations of opiate antagonists in various forms are generally known in the art.

Another approach to cocaine and nicotine addiction treatment is provided in U.S. Patent Application No. 20060286099, in which antibodies are produced within the system by injecting a drug-hapten carrier conjugate. When the addictive drug is taken, its effects are neutralized by the antibodies. A similar approach to cocaine addiction has been taken by the National Institute on Drug Abuse in a 2009 study where antibodies targeting cocaine were induced in a patient's body, thereby neutralizing the drug's effects, leading to the absence of drug-induced euphoria. One risk involved in this approach, in the case of addictive drugs such as cocaine, is uncontrolled overdosing by the patient to induce the drug's effects, which could be harmful or even fatal.

For individuals addicted to tobacco, nicotine preparations in various forms such as patches, gum or nasal spray and bupropion are available. An implant of biodegradable material or a patch containing lobeline for nicotine addiction is disclosed in U.S. Pat. No. 5,486,362. Among other addictions alcoholism is also treated with opioid antagonists and sometimes by surgery.

U.S. Pat. No. 7,172,767 discloses an oral dosage form of a pain management drug that contains opioid agonist/antagonist combinations. When a user overdoses on the oral dosage form, it induces unpleasant withdrawal symptoms of an addictive drug such as hydrocodone. However, the drug combination of this disclosure is an oral dosage form and is intended to minimize the abuse potential of a pain treatment composition, rather than for treatment of drug addiction.

A drawback of such addiction treatment regimens is the need for compliance by the patient. For effective treatment, patient involvement is essential in terms of identifying the correct dosage and compliance to the treatment regimen, both of which are often further assisted by behavioral therapy and social support programs. In the absence of one or more of these patient involvements, relapse is common. A particularly difficult problem in patient compliance is the unpleasant feeling that is the side effect of many de-addiction medications.

Furthermore, many such agents themselves produce unpleasant side effects that may vary depending on patient-related factors such as the agent dosage, patient body weight, age of the patient, and so on. For example, naltrexone may produce any of the following side effects: nausea, headache, dizziness, fatigue, insomnia, anxiety or sleepiness. Other agents such as methadone have similar side effects, but the severity may vary with each individual drug. Because of the side effects of these agents, their continued presence in the patient at all times is not totally desirable.

There is therefore a need for a method in which medication is delivered to individuals when required and without reliance on an action by the user to ensure compliance. It is one of the objects of the present invention to provide such a method of treatment. Another advantage of such treatment could be its use as a preventive in susceptible populations such as disadvantaged groups, particular social groups and at specific locations.

### SUMMARY OF THE INVENTION

The invention discloses a method of treatment for drug addiction, comprising administering a therapeutic agent to a patient. The therapeutic agent is delivered in an inactive state and is converted into its active form in the presence of a narcotic. The conversion produces at least one unpleasant side effect such as nausea, vomiting, unpleasant taste and headache for the patient. Additionally, the therapeutic agent may be configured to block or decrease the effect of the narcotic by blocking or competitively binding the narcotic's receptors within the body. The therapeutic agent is implanted in the form of a depot, and may be a prodrug, or microspheres. The agent may be injectable or may be packaged in the form of bioerodible sticks. The implant may also be delivered via a surgical method.

Furthermore, the invention discloses a method for determining whether an individual has consumed a banned substance such as a narcotic, comprising administering the therapeutic agent to a patient in an inactive state. The therapeutic agent is configured to convert into its active form in the presence of the banned substance. The active state of the therapeutic agent is configured to be detectable in the bodily fluid or hair sample of the patient, such that it can be determined whether an individual consumed the banned substance by analyzing the presence of the active form of the therapeutic agent in the bodily fluid or hair sample. Additionally, the therapeutic agent is configured to be substantially unaffected by masking agents such that the therapeutic agent may be converted to its active form and is detectable even in the presence of the masking agents.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 illustrates a method for treating drug addiction, in accordance with one embodiment of the present invention.

FIGS. 2A-C show an example of a therapeutic agent in a non-biodegradable encapsulation.

FIGS. 3A-C show an example of a therapeutic agent encapsulated in biodegradable form.

FIG. 4 shows an example where the therapeutic agent is in the form of dispersed, biodegradable microspheres.

### DETAILED DESCRIPTION

Although the detailed description contains many specifics, these should not be construed as limiting the scope of the invention but merely as illustrating different examples and aspects of the invention. It should be appreciated that the scope of the invention includes other embodiments not discussed in detail herein. Various other modifications, changes and variations which will be apparent to those skilled in the art may be made in the arrangement, operation and details of the method and apparatus of the present invention disclosed herein without departing from the spirit and scope of the invention as described here.

The invention is a method of treatment for drug addiction in which a therapeutic agent is administered to a patient such that it is present systemically in its inactive form. The therapeutic agent is triggered to convert to active form on ingestion or upon coming into contact with an addictive drug or banned substance, such as a narcotic. As referred herein, the term narcotic is meant to include opioids, such as opium and heroin, methamphetamine, cocaine (benzoylmethylecgonine), ketamine, MDMA (3,4-Methylenedioxymethamphetamine), lysergic acid diethylamide, or cannabinoids. Additionally, narcotic is meant to include alcohol, nicotine, or any other controlled substance.

The active form of the therapeutic agent produces unpleasant side effects for the patient. Optionally, the therapeutic agent is designed, in its active form, to facilitate breakdown and elimination of the narcotic substance from the body. The method of treatment envisages no voluntary action for activating the treatment, once the therapeutic substance, in its inactive form, is administered. Examples of the therapeutic agents or substances suitable for the invention include, but are not limited to: naltrexone, LAAM, methadone, etc, of which the prodrug form or the inactive form may be utilized.

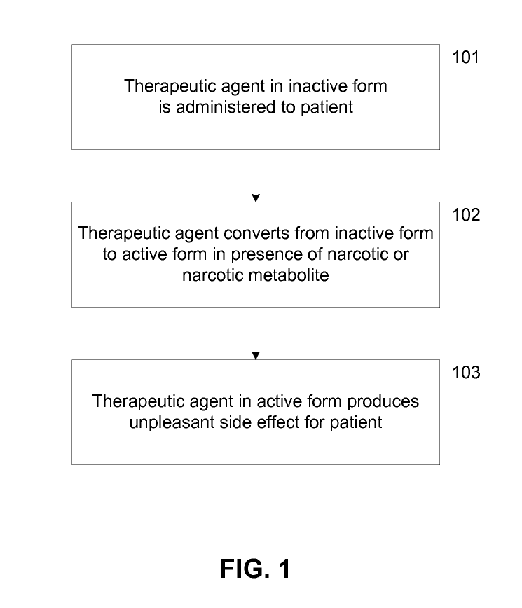

In its first embodiment, the invention discloses a method of treatment for drug addiction as shown in FIG. 1. A therapeutic agent is administered to a patient in step 101, wherein the agent is present in an inactive state. The physical form of the agent may be microspheres, a depot, or other forms that can provide sustained release of the agent. The agent remains inactive in the body of the patient until a narcotic is introduced into the system, which activates the agent. When a narcotic substance is ingested or systemically encountered in step 102, the therapeutic agent is converted into its active form by the presence of the narcotic. At step 103, the active form of the agent resulting from the conversion produces unpleasant side effects for the patient. Such unpleasant side effects include, but are not limited to: nausea, unpleasant taste, headache, vomiting, or other side effects. In one embodiment, in addition to producing unpleasant side effects, the therapeutic agent may be formulated to act as an antidote to the narcotic substance, for example by blocking the narcotic's receptors within the body. Optionally, the therapeutic agent is designed to aid elimination of the narcotic from the body through normal excretory channels, such as urine or perspiration.

In one embodiment, the therapeutic agent is envisaged to be a prodrug. The prodrug is converted into its active form on contact with the narcotic substance and/or on contact with a metabolite of the narcotic substance.

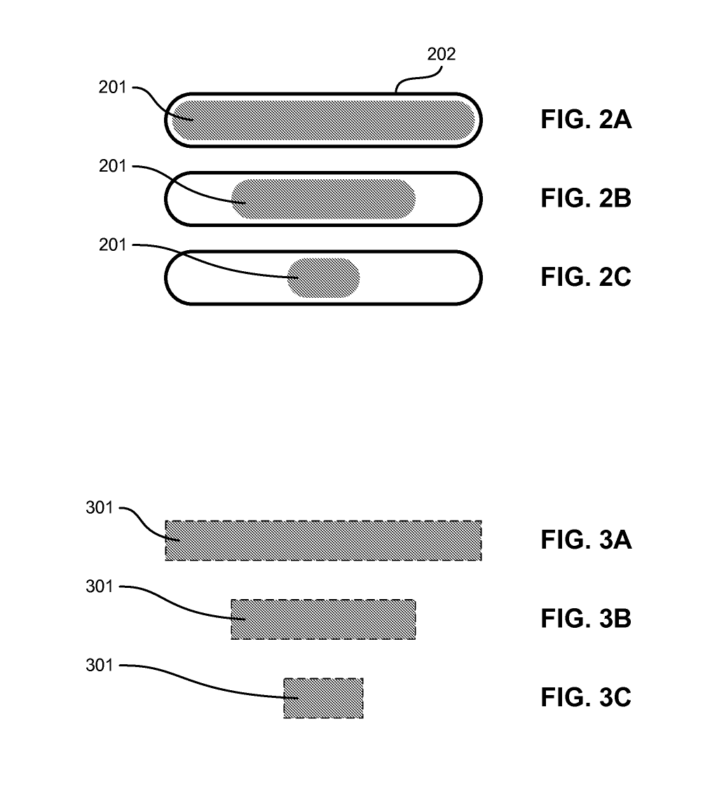

In another embodiment, the therapeutic agent is administered into the body in the form of an implant, for example as shown schematically in FIGS. 2A to 2C. As shown in FIG. 2A, the therapeutic agent 201 is housed within the body of an implant casing 202.

To facilitate dispersion of the therapeutic agent 201 into the patient's system the implant casing 202 optionally comprises pores to allow diffusion of the agent 201 therethrough, thereby depleting its content within the implant casing 202, as shown in the progression of FIGS. 2B and 2C.

The implant casing 202 is made of a non-biodegradable but biocompatible material, such as stainless steel or other corrosion resistant alloy, or a polymer. The implant is delivered subcutaneously and remains in the body while the therapeutic agent 201 is present. The implant casing 202 may be removed after the therapeutic agent 201 is depleted. The implant may be designed to remain within the patient's system for a convenient time period, for example 3-6 months, depending on the size of the implant and concentration of the therapeutic agent 201. The implant casing 202 may be shaped for convenient implantation and removal, as is well known in the art.

Optionally, the non-biodegradable implant is designed to carry an electronic chip such as an RFID device. The electronic chip can be used for detecting or monitoring the implant, for example in maintaining patient records and in scheduling a replacement when required. Furthermore, the implant might comprise circuitry that transmits a periodic signal to an external source that confirms the proper working of the implant. This would avoid tampering with the implant, such as the implantee attempting to remove the implant. Additionally, the implant is configured to detect the presence of the narcotic by detecting the active form of the therapeutic agent. Furthermore, the implant may comprises sensors that are configured to detect the presence of the narcotic or banned substance as defined below in situ when the presence of the narcotic or banned substance can be detected in a body fluid, such as interstitial fluid similar to implanted glucose sensing devices.

Alternatively, the therapeutic agent may be administered into the body as a biodegradable or bioerodible implant. For example, as shown schematically in FIGS. 3A-3C, the therapeutic agent 301 is mixed with a biodegradable matrix, such as a biodegradable polymer, such that dissolution of the matrix releases the therapeutic agent 301. As shown in FIGS. 3B and 3C, the amount of therapeutic agent 301 remaining within the implant depletes with time. The implant may be shaped and sized or packaged conveniently for implantation, for example in the shape of bioerodible sticks or rods. The concentration of the therapeutic agent 301 and characteristics of the biodegradable matrix may be tailored such that the implant remains active within the body for a convenient duration of time, for example 3-6 months.

Additionally, the therapeutic agent may be administered into the body in a therapeutic dosage form. The therapeutic dosage form may be bioerodible when implanted in the body. Generally, the therapeutic dosage form is configured as a controlled delivery system. The dosage form permits the therapeutic agent to be delivered to the systemic circulation of the patient in a controlled manner to ensure that the therapeutic agent is not delivered in a dosage that is harmful to the patient.

In one embodiment, the therapeutic dosage form is at least one reservoir device in which the therapeutic agent forms a core surrounded by an inert diffusion barrier. The reservoir device includes membranes, capsules, microcapsules, liposomes, and hollow fibers.

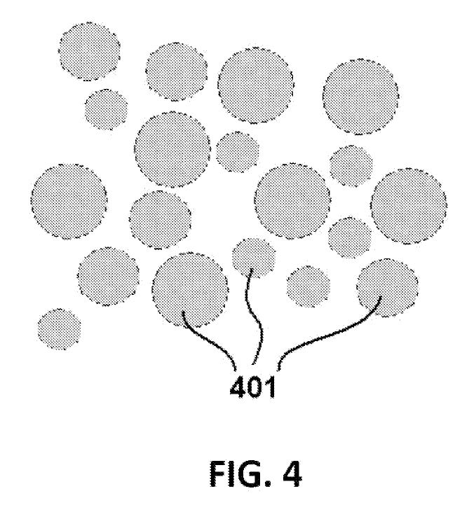

In another embodiment, the therapeutic dosage form is one or more microspheres. The microspheres are generally prepared from natural and/or synthesized polymers that act as miniature time-release drug capsules. The microspheres comprising the therapeutic agent may be injected within the body, dispersed within the body, and thereafter the therapeutic agent may be time-released within the body over a period. As shown in FIG. 4, the microspheres 401 may be of different sizes and dissolution characteristics to provide for a controlled and continual supply of the therapeutic agent for a predetermined time period. This method has the advantage that the therapeutic agent's presence is unobtrusive and cannot be easily removed by the patient

The biodegradable implants are advantageous in that they are eventually absorbed by the body and thus need not be removed surgically. Furthermore, the bioerodible implants in the form of microspheres or other similar dosage forms, have the additional advantage of being hard to extirpate from the implant site by the patient. Addicts are known to take extreme measures to satisfy their craving and a non-biodegradable, contained dosage form might be susceptible to the addict's attempts to remove the implant from the body.

The implants (both biodegradable and non-biodegradable) may be placed within the body at any convenient location in the body. The implants may be inserted in an easily accessible location close to the skin, or within a body orifice using a needle of suitable size. Alternatively, the implants may be inserted in place by a more elaborate surgical technique if it is desired to locate the implants deeper within the body.

The present invention is envisaged to be useful in managing treatment of addiction and preventing relapse. Apart from the therapeutic agent inducing severe negative side-effects when converted into its active form in the presence of a narcotic, the therapeutic agent can be chosen to be an indicator for narcotic consumption. For this purpose, in one embodiment the patient's urine or hair can be tested for the presence of the active form of the therapeutic agent, even when the narcotic itself is not detectable, for example because of its prior elimination from the patient's system. This may be achieved by designing the therapeutic agent in active form to remain in the system for a longer time period than the narcotic. If such detection is positive, that would reveal that the narcotic had been ingested, which in turn would allow other actions to be taken, such as prevention of further access to the narcotic, administration of the therapeutic agent in higher dosage, imposition of restrictive or punitive measures, etc.

Another embodiment of the invention relates to identifying whether an individual has consumed a banned substance. The identification of whether an individual has consumed a banned substance is advantageous in preventing use of performance enhancement substances that provide unfair advantage in sporting competitions, managing treatment of addiction, preventing relapse, and aiding authorities in identifying users of banned substances. As referred to herein, the term banned substance is meant to include any narcotic as defined above. The term banned substance is further meant to include performance enhancing substances such as anabolic steroids, beta-2-agonists, human growth hormone, amphetamines, or erythropoietin. Additionally, the term banned substance is meant to include any substance that is banned or illegal for consumption by an individual affiliated with any groups, associations, and/or jurisdictions within the United States, or any other countries.

As part of this determination, an inactive form of a therapeutic agent is administered to the individual. The therapeutic agent is converted to an active form in the presence of a banned substance that is consumed by the individual. The therapeutic agent can be chosen such that the active form of the therapeutic agent has a long half-life. Depending on the situation, the half-life could range from days to many months. In one embodiment, the half-life of the therapeutic agent is configured to be longer than a half-life of the banned substance such that the active form of the therapeutic agent remains in the system for a longer time period than the banned substance.

A test revealing the presence of the active form of the therapeutic agent in the body of the individual would imply that a banned substance was consumed. The advantage of using an agent with a long half-life is to identify individuals who had consumed a banned substance, and had the eliminated from their system. For example, urine, hair or saliva of a patient may be tested for the presence of the active form of the therapeutic agent, even when the banned substance itself is not detectable because of its prior elimination from the patient's system.

Furthermore, the therapeutic agent is configured to convert to an active form even if one or more masking agent, such as epitestosterone designed to prevent the detection of the banned substance is present in an individual along with the banned substance. The active form of the therapeutic agent is configured to be substantially unaffected by the masking agent such that the active form is detectible in an individual even if the banned substance is rendered undetectable by the masking agent.

When a detection of the presence of the active form of the therapeutic agent is positive, it would reveal that the banned substance had been consumed. Such information would allow other actions to be taken, such as prevention of further access to the banned substance, administration of antidotes, alert the proper authorities, imposition of restrictive or punitive measures, etc.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method of treatment for drug addiction, comprising:
administering a therapeutic agent to a patient, wherein the therapeutic agent is in an inactive state;
wherein the therapeutic agent is converted into its active form in the presence of a narcotic.

2. The method of claim 1, wherein the conversion produces at least one unpleasant side effect for the patient.

3. The method of claim 2, wherein the side effect comprises one or more of nausea, vomiting, unpleasant taste and headache.

4. The method of claim 1, wherein the therapeutic agent is implanted in the form of a depot.

5. The method of claim 1, wherein the therapeutic agent is a prodrug.

6. The method of claim 4, wherein the depot comprises microspheres.

7. The method of claim 4, wherein the depot comprises insertable bioerodible sticks.

8. The method of claim 4, wherein the implant is delivered subcutaneously using a needle.

9. The method of claim 4, wherein the implant is delivered into the body by surgical methods.

10. The method of claim 1, wherein the therapeutic agent is configured to block an effect of the narcotic.

11. A method of determining whether an individual has consumed a banned substance, comprising:
administering a therapeutic agent to the individual, wherein the therapeutic agent is administered in an inactive state and wherein the therapeutic agent is converted into its active form in the presence of the banned substance; and
analyzing a bodily fluid to determine existence of the active form of the therapeutic agent.

12. The method of claim 11, further comprising determining whether the individual has consumed a banned substance based on the existence of the active form of the therapeutic agent in bodily fluid of the individual.

13. The method of claim 11, further comprising determining whether the individual has consumed a banned substance based on the presence of the active form of the therapeutic agent in hair samples of the individual.

14. The method of claim 11, wherein the active form of the therapeutic agent is unaffected by one or more masking agents.

15. The method of claim 14, further comprising determining whether the individual has consumed a banned substance based on the presence of the active form of the therapeutic agent in the individual.

16. A therapeutic dosage form, comprising:
a therapeutic agent in an inactive form, wherein the therapeutic agent is configured to be converted into its active form in the presence of a banned substance consumed by an individual.

17. The therapeutic dosage form of claim 16, wherein the dosage form is configured as a controlled delivery system, and permits the therapeutic agent to be delivered to a systemic circulation of the individual in a controlled manner.

18. The therapeutic dosage form of claim 17, wherein the dosage form is bioerodible when implanted in the individual.

19. The therapeutic dosage form of claim 17, wherein the dosage form is one or more injectable microspheres.
